(19) United States
(12) Patent Application Publication
(10) Pub. No.: US 2005/0037033 A1
(43) Pub. Date: Feb. 17, 2005

(54) Microbicidal compositions and methods and use

(75) Inventors: Florence Camus-Bablon, Seattle, WA (US); Lourens J.D. Zaneveld, Sao Paulo (BR); Donald Waller, Chicago, IL (US)

Correspondence Address: STERNE, KESSLER, GOLDSTEIN & FOX PLLC, 1100 NEW YORK AVENUE, N.W., WASHINGTON, DC, 20005, US

(73) Assignee: Program for appropriate Technology in Health, Seattle, WA (US)

(21) Appl. No.: 10/850,456

(22) Filed: May 21, 2004

Related U.S. Application Data
(60) Provisional application No. 60/472,988, filed on May 23, 2003.

(51) Int. Cl. A61K 31/44

(52) U.S. Cl. 424/400; 514/345

(57) ABSTRACT

The present invention is directed to microbicidal compositions containing a microbicidal formulation or agent and methods of use thereof for preventing the transmission of or treating sexually transmitted infections and/or common vaginal infections, while minimizing disruptions to vaginal ecology and epithelium. In a preferred embodiment, the microbicidal formulation or agent is ciclopirox olamine. These compositions are preferably encapsulated in the form of a foam, cream, wash, gel, suppository, ovule, lotion, ointment, film, tablet, foaming tablet, tampon, vaginal spray, or aerosol. Preferably the concentration of microbicidal agent is approximately 0.01% to approximately 50% by weight, more preferably between approximately 0.1% to approximately 28%, and most preferably between approximately 0.5% and approximately 5%.

### BACKGROUND

1. Field of the Invention

The present invention relates generally to a composition and method for the prevention or treatment of sexually transmitted infections and/or common vaginal infections, and more specifically to a microbicidal composition containing a microbicidal formulation or agent and the use thereof.

2. Description of the Related Art

Sexually transmitted infections (STIs), referring to infections that are most often transmitted by direct sexual contact, remain an increasingly serious worldwide public health problem. These STIs, particularly viral infections, present a public health crisis.

Women are especially at risk as they are more susceptible to infection, many STIs are asymptomatic and there is a high morbidity rate associated with untreated infections.

Since its recognition in 1981, the acquired immunodeficiency syndrome (AIDS) has become a catastrophic pandemic. The AIDS pandemic is a premiere public health concern. Individuals who are at high risk of HIV/AIDS infection are also at risk of infection by other sexually transmitted pathogens. Similarly, individuals at risk for non-HIV/AIDS sexually transmitted pathogens are also at high risk for HIV/AIDS infection.

Additionally, it is significant to note that women comprise the most rapidly increasing population of the AIDS epidemic. Sexual transmission of HIV/AIDS in women occurs by infected semen being placed into the vagina, rectum, or other orifice. Currently, the only prevention strategy available for HIV/AIDS prevention is the condom or abstinence.

Clinical pathologies attributable to STIs are profound. STIs cause acute and chronic infections, infertility, and in some cases cancer. Vaccines, which are costly and time-consuming to develop, are unavailable for STI/AIDS prevention. HIV/AIDS treatment employs therapeutic strategies, such as retrovirus triple therapy (e.g., AZT, DDI, etc.) to lower virus burden. However, the high expense of treatment renders this therapeutic option practically unavailable to populations in developing countries where HIV/AIDS is most prevalent. Indeed, the sum of all available STI/AIDS therapeutics is effective against only a limited number of susceptible pathogens. Furthermore, this limited therapeutic arsenal is largely confined to proprietary formulations, which are costly for the afflicted to procure.

Common vaginal infections also pose an increasingly serious worldwide public health problem and can increase the risk of acquiring HIV/AIDS and other STIs. Vaginal candidiasis is the most common form of vaginitis, occurring more frequently than trichophyton, chlamydia, gonorrhea, or other bacterial infections. It is estimated that 75% of women will experience at least one episode of vulvovaginal candidiasis in their lifetime. Forty to 50% will experience a second episode in their life time. A much smaller (probably less than 5%), but still significant, number of women will suffer from repeated, often intractable attacks. Candidiasis is known to increase the risk of HIV/AIDS acquisition. Bacterial vaginosis (BV), previously known as nonspecific vaginitis or Gardnerella vaginitis, is the most common cause of vaginal discharge. It may be the cause of up to 50% of cases of vaginitis in all women and from 10-30% in pregnant women. BV is not a sexually transmitted disease although it is sometimes listed as one. However, the risk of contracting the disease increases with multiple sex partners. Although treatment is available for these diseases, methods to prevent them and improved methods of treatment are still needed.

Presently marketed vaginal contraceptive compositions, often containing nonoxynol-9 as an active ingredient, are generally known in the art. While presently marketed vaginal contraceptive formulations aid in preventing pregnancy, their ability to effectively prevent STIs, particularly HIV/AIDS as well as oral, rectal and vaginal infections, is very limited. Nonoxynol-9 and other detergents as well as their compositions can destroy the natural and safe ecology of the vagina, such as by inactivating lactobacillus bacteria. Further, spermicides may cause vaginal irritation, particularly with frequent exposure or higher doses. Recent analyses show that nonoxynol-9, when used frequently by women at high risk, may increase the risk of HIV infection (WHO 2002, WHO/CONRAD technical consultation on nonoxynol-9, Geneva).

Accordingly, there remains an urgent and compelling need for alternative methods and compositions to prevent the transmission of sexually transmitted infections, particularly HIV/AIDS, and common vaginal infections, while minimizing vaginal disruptions.

### SUMMARY OF THE INVENTION

To achieve the foregoing, and in accordance with the purposes of the present invention as embodied and broadly described herein, it is an object of this invention to provide microbicidal compositions that prevent the transmission of or treat sexually transmitted infections including HIV/AIDS and/or common vaginal infections while minimizing disruptions to vaginal ecology and epithelium.

In one aspect, the present invention includes vaginal microbicidal compositions including microbicidal formulations or agents suitable for preventing the transmission of sexually transmitted infections comprising the microbicidal formulations or agents of the present invention. Another embodiment of the present invention includes microbicidal compositions suitable for preventing the transmission of common vaginal infections comprising the microbicidal formulations or agents of the present invention. A further embodiment of the present invention includes microbicidal compositions suitable for treating sexually transmitted infections comprising the microbicidal formulations or agents of the present invention. These compositions are preferably provided in the form of a foam, cream, wash, gel, suppository, ovule, lotion, ointment, film, tablet, foaming tablet, tampon, vaginal spray, aerosol or other base or carrier.

In a preferred embodiment, the microbicidal formulation or agent is ciclopirox olamine. The concentration of the microbicidal agent will vary depending on the base or carrier. Preferably the concentration will fall within the parameters of approximately 0.01% to approximately 50% by weight, more preferably between approximately 0.1% to approximately 28%, and most preferably between approximately 0.5% and approximately 5%. In a preferred embodiment, the base or carrier is a gel or cream and the microbicidal agent concentration will preferably range from approximately 0.5% to approximately 5% by weight.

The present invention may further include methods of preventing conception and transmission of sexually transmitted infections by using microbicidal compositions according to the present invention by themselves or in conjunction with condoms, delivery devices, applicators, barrier-type devices and other vaginal or anorectal compositions.

### DETAILED DESCRIPTION OF THE PREFERRED EMBODIMENTS

A preferred embodiment of the present invention is now described. While specific configurations and arrangements are discussed, it should be understood that this is done for illustrative purposes only. A person skilled in the relevant art will recognize that other configurations and arrangements can be used without departing from the spirit and scope of the invention. It will be apparent to a person skilled in the relevant art that this invention can also be employed in a variety of other devices and applications.

According to the present invention, the protection from sexually transmitted infections, such as HIV/AIDS, and common vaginal infections, such as bacterial vaginosis and vaginal candidiasis, can be obtained by placing an antimicrobial composition or device in the vagina, rectum or other orifice, which can inactivate the virus, prevent or limit contact of the virus or its carrier cells with the epithelium or prevent or hinder its entry into the orifice.

According to the present invention, microbicidal compositions have been found to be useful for protection against and prevention of sexually transmitted infections including HIV/AIDS. They may be used alone or in conjunction with delivery and/or contraceptive devices or methods, such as mechanical barrier-type devices (such as a diaphragm, cap, or sponge), vaginal contraceptives, vaginal ring, intra-uterine-devices, rhythm, and a variety of applicators. Microbicidal compositions according to the present invention generally contain a microbicidal formulation or agent and a base or carrier, such as a foam, cream, wash, gel, suppository, ovule, lotion, ointment, film, tablet, foaming tablet, tampon, vaginal spray, or aerosol.

Microbicidal agents and formulations act according to a variety of mechanisms. Specifically, such agents and formulations may destroy microbes, prevent their pathogenic action, inhibit their entry into or replication in cells, prevent tissue entry, or inhibit their growth. In particular, non-cytotoxic microbicides act by preventing interactions of STI-causing viruses and bacteria with host cells, or otherwise prevent contact with host cells. Microbicidal agents and formulations, also referred to as anti-infective agents and formulations, may be applied to the body cavities such as the vagina and rectum. For other anti-infective use, they may also be applied to the skin, mucous membranes, and orally. Topical microbicidal agents and formulations may be directed at bacteria, viruses, fungi, and parasites. Such topical microbicidal agents and formulations are convenient for vaginal or rectal application and have been successfully employed in the prevention and treatment of a number of infections including some STIs in animal models. Preferable microbicidal agents and formulations inactivate bacteria and viruses, and are inexpensive, affordable, stable at ambient temperature, compatible and active after mixture with cosmetically acceptable formulations, non-toxic and non-damaging to vulvar, vaginal, cervical, penile or other epithelium.

The present invention provides microbicidal compounds and microbicidal formulations or agents that prevent the transmission of or treat sexually transmitted infections and/or common vaginal infections. Sexually transmitted infections include, but are not limited to, HIV/AIDS, herpes (caused by herpes simplex virus type 1 (HSV-1) or herpes simplex virus type 2 (HSV-2)), gonorrhea, chlamydia, syphilis, and trichomoniasis. Common vaginal infections include, but are not limited to, bacterial vaginosis (BV) and vaginal candidiasis. Similar microbicidal compositions and methods of application of such compositions, as described herein, can be used for treating sexually transmitted infections and/or common vaginal infections and for preventing the transmission of sexually transmitted infections and/or common vaginal infections.

The composition of the present invention is a microbicidal composition including a microbicidal formulation or agent. In a preferred embodiment the microbicidal composition may further include a base or carrier, such as a foam, cream, wash, gel, suppository, ovule, lotion, ointment, film, tablet, foaming tablet, tampon, vaginal spray, aerosol, or other base or carrier as would be apparent to one skilled in the relevant art.

In a preferred embodiment, the microbicidal formulation or agent is a hydroxypyridone antimycotic agent. Derivitives of hydroxypyridones include, but are not limited to, compounds such as ciclopirox olamine, mimosine, and deferiprone. Hydropyridones are described in the following publications, which are hereby incorporated by reference in their entirety (Korting HC, Grundmann-Kollman M, The hydroxypyridones: a class of antimycotics of its own. Mycoses 1997; 40: 243-247; Lohaus G, Dittmar W, The chemistry of antimicrobial active 1-hydroxy-2-pyridones. Drug Res 1981; 31: 1311-13-16).

In a preferred embodiment of the present invention, the microbicidal agent or formulation of the present invention may include ciclopirox olamine. Ciclopirox olamine is a broad-spectrum hydroxypyridone antimycotic and antibacterial agent. It inhibits the growth of pathogenic dermatophytes, yeasts and Malassezia furfur. Ciclopirox olamine exhibits fungicidal activity in vitro against isolates of Trichophyton rubrum, Trichophyton mentagrophytes, Epidermophyton floccosum, Microsporum canis and Candida albicans. It prevents the apoptotic death of neuronal cells brought about by trauma or stroke. In a preferred embodiment, the ciclopirox olamine of the present invention is a white crystal powder having a molecular formula of C₁₂H₁₇NO₂ C₂H₇NO and a CAS Number of 41621-49-2. Synonyms, derivatives and marked products containing ciclopirox olamine include: 6-Cyclohexyl-1-hydroxy-4-methyl-2-(1H)-pyridone ethanolamine salt; Ethanolamine salt; HOE-296; Batrafen; Brumixol; Ciclochem; Dafnegin; Loprox; Microxolamina; Mycoter; and Terit. Ciclopirox olamine is available from sources such as Micro Labs Limited, P.O. Box No. 5061, 3 Queens Road, Bangalor 560 001. Ciclopirox olamine is used in various formulations including Loprox from Aventis Pharma Ltd., Aventis House, 50 Kings Hill Avenue, West Malling, Kent ME19 4AH; Dafnegin from CSC Pharmaceuticals; Dafnegil from Monsanto Italiana S.p.A.; Dafnegin from Pharmacia Italia S.p.A.; Micoxolamina from Mastelli s.r.l.; Brumixol from Bruschettini S.r.l.; Micomicen from Sanofi-Synthelabo S.p.A.; Micopirox from Master Pharma SRL; Olamin from Micro Labs Limited Bangalore; Fungowas from Chiesi Espana; Ciclochem Vaginal from Novag; Canolen Vaginal Cream from BioFarma; Biroxol from Salus Researchers SPA; and Aquomin Vaginal from Belmac (and is also available from Lyka Labs Ltd., 77 Nehru Road, Vile Parle (East), Mumbai, 400099, INDIA). Ciclopirox olamine is further described in U.S. Pat. No. 3,883,545, which is incorporated herein by reference.

In an alternate embodiment, the microbicidal agent or formulation of the present invention may include Lecithin. Lecithin is a lipid material belonging to the class known as phospholipids, and is manufactured in the body by choline and inositol. Lecithin is high in phosphorous and unites with iron, iodine and calcium. Lecithin is utilized in a wide variety of food and industrial applications. Formulations including Lecithin are available including Gynecure, available from Pfizer Products, Inc., 235 East 42^{nd} Street New York, N.Y. 10017; Monistat, available from Johnson & Johnson, One Johnson & Johnson Plaza, New Brunswick, N.J. 08933; Livage, available from Aristo Pharmaceuticals Ltd., P.O. Box 5554, Ktm, Maitighar, Kathmandu, NEPAL; and Sultrin available from Ortho-McNeil Pharmaceutical, Inc., 1000 Route 202 South, Raritan, N.J. 08869.

In an alternate embodiment, the microbicidal agent or formulation of the present invention is Fain Gel. Fain Gel is available from Applied Pharma Research (APR) and Elder Pharmaceuticals Ltd., D-220, T. T. C. Industrial Area, Navi Mumbai 400706, INDIA. Fain Gel includes asiaticoside, sodium hyaluronate, and carrageenan. The active ingredient is asiaticoside, a substance having excellent healing properties and obtained from the plant Centella Asiatica. Asiaticoside stimulates the activity of fibroblasts, helping to synthesize collagen fibers and accelerate healing processes.

Sodium hyaluronate is a glycosaminoglycan which has a very high molecular weight (500,000). Glycosaminoglycans are also called as mucopolysaccharides because they contain long chains of disaccharide units. These disaccharide units are formed by a monosaccharide and by an amino sugar linked together by glycosidic bridges. Sodium hyaluronate has a capacity to absorb water and swell, making it particularly useful as a hydrating lubricating agent. Sodium hyaluronate distributes itself on the surface of the cutaneous layer, obstructing the loss of water, and thereby hydrating the skin.

Carrageenan is added as an excipient to Fain Gel as thickening and gelling agent. Carrageenan is a polysaccharide sulphonate extracted from algae belonging to rhodophyceae. Carrageenan contributes to the hydrating and lubricating action of Fain Gel.

Fain Gel has been shown to relieve problems related to Dyspareunia and Vaginismus, and improves and facilitates wound healing in patients after undergoing gynecological surgery. Fain Gel provides cosmetic benefits, and provides immediate lubricating action; causes hydration and trophism of vaginal mucosa without altering the bacterial flora or the pH of the vaginal mucosa; facilitates recovery from vaginal disease; accelerates wound healing without scar formation; and is well tolerated and is easy to apply.

Fain Gel helps normalize dryness in the feminine external genital region. In particular, it has been shown to mitigate the irritating processes occurring during the menstrual cycle. Fain Gel leaves the area moisturized for a long time while maintaining the physiological pH. The long-lasting hydrating activity improves the trophism of the genital mucosa and facilitates its recovery from paraphysiologic or pathologic conditions.

In an alternate embodiment, the microbicidal agent or formulation of the present invention may include Moiste. Moiste is a multi-purpose wheat-germ oil aloe comprising aloe, wheat germ, honey, and tea tree oil. In a preferred embodiment, Moiste consists of 10% aloe vera, 0.6% wheat germ oil, 1% honey, 0.1% tea trea oil, and further contains Vitamin E. Components of all four have shown to possess antibacterial and antifungal properties. Aloe is the dried juice of leaves of A. barbadensis (known as curacao aloes), or of A. perryi (known as socotrine aloes), or of A. africana and A. spicata (known as cape aloes). All Aloe species contain anthraquinone glycosides. Barbaloin (aloe-emodin anthrone C-10 glucoside) is the major active constituent. Aloe also contains isobarbalin, aloe-emodin, resins, aloetic acid, homonataloin aloe-sone, chrysophanic acid, chrysamminic acid, galactouronic acid, choline choline salicylate, saponins, mucopolysacchrides, glucosamines, hexuranic acid, and coniferyl alchohol. A major carbohydrate fraction of aloe vera is acemannan. Wheat germ contains agglutinins.

Moiste is available from Genix Pharma Ltd. H.No. 8-3-166/7/2, Erragadda, Hyderabad—500 018, INDIA; and is also available from Hetero Drugs, Hetero House, H. No. 8-3-166/7/1, Erragadda, Hyderabad—500 018, INDIA.

In alternate embodiments of the present invention the microbicidal agents and formulations may include Aminacrine, Gramicidin, Hamycin, Lactacyd Feminine Wash, Lansoprazole, Amrut Malam, Dermal (Amrutanjan Pain Balm), Emolene Cream, Pama Malam, Myron, and V-Gel.

In one embodiment, the microbicidal agent or formulation of the present invention may include Hamycin. Hamycin is a polyene antibiotic complex produced by Streptomyces imprina. It is an anti-fingal that has been used in the treatment of candidiasis and otomycosis. Hamycin is available from Hindustan Antibiotics Limited, Pimpri, Pune 411018, INDIA.

In an alternate embodiment, the microbicidal agent or formulation of the present invention may include Lactacyd Feminine Wash. Lactacyd Feminine Wash is a feminine hygiene product containing natural and mild cleansing ingredients extracted from milk. Lactacyd includes lactic acid, and is available from GlaxoSmithKline, Brentford, TW8 9GS, United Kingdom. In a preferred embodiment, the Lactacyd contains lactoserum (whey) and lactic acid, and has a pH of approximately 3.5. Preferably the Lactacyd contains the proteins alpha-lactoglobulin, beta-lactoglobulin, bovine serum albumin and lactoferrin (Lf). Lactoferrin is present in exocrine secretions that are commonly exposed to microbes such as milk, tears, nasal exudates, saliva, bronchial mucus, gastrointenstinal fluids, cervico-vaginal mucus and seminal fluids. Lactoferrin is a major constituent of the secondary specific granules of circulating polymorphonuclear neutrophils (PMNs). Lactoferrin is antiviral, antibacterial, antimycotic, anti-inflammatory, antineoplastic and iron-binding, and has shown therapeutic effects in cancer, HIV/AIDS, cytomegalovirus, herpes, chronic fatigue syndrome, candida albicans and other infections.

In an alternate embodiment, the microbicidal agent or formulation of the present invention may include Aminacrine, also known as amicarine, aminoacridine, 9-acridinamine, aminacrine hydrochloride, aminoacridine hydrochloride, 5-aminoacridine hydrochloride, monacrin, and monacrin hydrochloride. Aminacrine is a broad spectrum antimicrobial drug that is known to have been used as an antiseptic in vaginal and excocervical infections, such as candidiasis, trichomoniasis or haemophilus, and as further been used as a prophylactic agent in gynecological procedures. In a preferred embodiment, the Aminacrine of the present invention is a yellow powder having a molecular formula of C₁₃H₁₀N₂ HCl and a CAS Number of 52417-22-8. Aminacrine is available from Clay-Parks Labs, 1700 Bathgate Avenue, Bronx, N.Y. 10457.

In an alternate embodiment, the microbicidal agent or formulation of the present invention may include Gramicidin. Gramicidin is a linear polypeptide antibiotic derived from Bacillus brevis (aneurinolyticus) soil bacteria with antiviral, antibacterial and antifingal properties. It is used in a wide variety of topical products worldwide, and is often mixed with antibiotics. It is spermatostatic but not spermicidal, and has been used as a vaginal contraceptive. Gramicidin has been shown to be a very effective HIV/AIDS and HSV inhibitory agent. Gramicidin is available from Glenmark Pharmaceuticals Ltd., B/2, Mahalaxmi Chambers, 22, Bhulabhai Desai Road, Mumbai 400 026.

In an alternate embodiment, the microbicidal formulation or agent of the present invention may include Lansoprazole. Lansoprazole is in a class of drugs called proton pump inhibitors (PPI) which block the production of acid by the stomach. Other drugs in the same class include rabeprazole (Aciphex), omeprazole (Prilosec) and pantoprazole (Protonix). Lansoprazole is also known as A-65006, AG-1749, Agopton, Lanzory, Ogast, Takepron, and Zoton. Lansoprazole is a substituted benzimidazole-2[[[3-methyl-4-(2,2,2-trifluorethoxy)-2-pyridyl]]methyl]sulfonyl]benzimidazole, having a chemical formula of C₁₆H₁₄F₃N₃O₂S.

In an alternate embodiment, the microbicidal formulation or agent of the present invention may include Amrut Malam, also known as Amrut Malham. Amrut Malam is an antiseptic product, primarily used for cracked skin. In a preferred embodiment, a 10 gram of ointment contains 2 grams of Garcina indica, 400 mgs of Ricinum communis (castor bean), 100 mgs of Karpoora (Cinnamonium camphora; camphor), 40 mgs of Shakhajira, 40 mgs of Gandhaka, (sulfur), 20 mgs of Pudina phool (Mentha piperita; peppermint), 40 mgs of Pushpanjan (zinc oxide), and a base. The major organic acid in leaves and rinds of Garcina indica is (−)-hydroxycitric acid. This plant also contains garcinol, a polyisoprenolated benzophenone. Amrut Malam is available from Amrut Pharmaceutical.

In an alternate embodiment, the microbicidal formulation or agent of the present invention may include Dermal, also known as Amrutanjan Pain Balm. Dermal is a skin ointment that includes 10% Kapoor powder (camphor); 1% Ajowan ka phool (biship's weed); 6% Pudina ka phool (menthol); 8% Wintergreen; 7% Turpentine; 14% Nilgriri (Eucalyptus oil); 1.5% Pudina (mint); 1% Ganjni-Citronela (castellano), Cymbopogon nardus, Gramineas (family), and Guchcha (sanskrit); and further includes Chaha (hari chaha: lemon grass) and Dal chini (cinnamon).

In an alternate embodiment, the microbicidal formulation or agent of the present invention may include Emolene Cream. Emolene cream is a moisturizing cream for treating dry skin. In a preferred embodiment, a 1 gram composition of Emolene cream includes 5 mgs of Dimethicone (a silicone derived oil), 1.5 mgs of Diazolindnyl urea, 15 mgs of Propylene glycol, and 1 mg of Lecithin. Emolene Cream is available from Schering-Plough Corporation, World Headquarters, 2000 Galloping Hill Road, Kenilworth, N.J. 07033.

In an alternate embodiment, the microbicidal agent or formulation of the present invention may include Myron. In a preferred embodiment, Myron contains Hirabol (Myrrh), Guggul, Dhavadi Flower, Bang Bhasma (tin ash), Ardusi leaves, Adrusi ghan, Abhrak Bhasma (mica ash), Lodhar (Symplocos racemosa), Rasavanti, Kasis Bhasma (ferrous sulphate ash), and Shilajit (complex mixture of organic & inorganic compounds). In an alternate embodiment, the composition of Myron includes 75 mgs of Hirabol, 15 mgs of Lodhar, 15 mgs of Dhavidi Flower, 15 mgs of Shilajit, 7.5 mgs of Vasaka Ghan (Adhatoda vasica), 30 mgs of Guggul (Commiphora Mukul), 15 mgs of Bang Bhasma, 7.5 mgs of Abrak Bhasma, 7.5 mgs of Vasaka Leaves (Adhatoda vasica), 7.5 mgs of Rasavanti, and 30 mgs of Kasis Bhasma. Myron vaginal tablets are used for the treatment of cervicitis, endometriosis, pruritus vulvae and pelvic inflammatory diseases, and for leucorrhoea and dysuria. Myron is available from Alarsin Pharmaceuticals, Mumbai, India.

In an alternate embodiment, the microbicidal agent or formulation of the present invention may include Pama Malam. In a preferred embodiment, a 10 gram composition of Pama Malam contains 250 mgs of Harital godanti (hartalaka; sulfur of arsenic), 500 mgs of Gandhak (sulfur), 125 mgs of Manasheela, 1 gm of Shankajira, 250 mgs of Kattha, 250 mgs of Kapoor (camphor), and further includes a vaseline base. Pama Malam is available from Amrut Pharmaceuticals.

In an alternate embodiment, the microbicidal agent or formulation of the present invention may include V-Gel. V-gel is a vaginal formulation known to be used for the treatment of fungal, trichomonal and bacterial vaginitis, to relieve symptoms of senile vaginitis, and to provide relief from vaginal itching, burning and foul-smelling discharge. In a preferred embodiment, the V-Gel of the present invention has the following composition: Triphala (4 mg), Satapatri (Rosa damascena; R. centifolia) (3.6 mg), Ela (elettaria cardamomum) (3.6 mg), Pumava (Boerhavia diffusa) (3.6 mg), Shaileyam (Parmelia perlata) (2.0 mg), Nirgundi (Vitex negundo) (1.6 mg), Haridra (Curcuma longa) (1.6 mg). V-Gel is available from The Himalaya Drug Company, Makali, Bangalore, 562-123 INDIA.

Other possible microbicidal agents and formulations according to the present invention include Nomarks cream, Pilex cream, Clearasil Ultra, Ketaconazole cream, Mylol and Zinc Undecenoate.

In an alternate embodiment, the microbicidal formulation or agent of the present invention may include Nomarks cream. Nomarks cream is a herbal preparation for clearing scars and marks, and includes wheat germ oil, haldi, chandan, tulsi and aloe, and is available from Ozone Ayurvedics, 1, L.S.C., Block A-3, Janak puri, New Delhi-110058, INDIA.

In an alternate embodiment, the microbicidal formulation or agent of the present invention may include Pilex Cream. Pilex cream is a natural, herbal formulation containing Calendula Officinalis (Zergul), Mimosa Pudica (sensitive plant, Vitex Negundo (five-leafed chaste tree), Eclipta Alba (Thisles), and Yashad Bhasma (Zinc Oxide). Pilex Cream is available from Body Health Products, Sapco, Box 398, Fort Road, St. Peter Port, Guernsey GY13FT.

In an alternate embodiment, the microbicidal formulation or agent of the present invention may include Clearasil Ultra Cream, including the active ingredients benzoyl peroxide and bentonite, and is available from Proctor and Gamble, 1 Procter & Gamble Plaza, Cincinnati, Ohio 45202.

In an alternate embodiment, the microbicidal formulation or agent of the present invention may include Ketoconazole cream. Ketoconazole cream contains ketoconazole, propylene glycol, cetyl alcohol, stearyl alcohol, isopropyl myristate, sorbitan monostearate, polysorbate, sodium solfite, and purified water. Ketoconazole cream is available from TEVA Pharmaceutical Industries Ltd., 5 Basel Street, Petach Tikva 49131, ISRAEL.

In an alternate embodiment, the microbicidal formulation or agent of the present invention may include Mylol. Mylol contains diethyl toluamide (DEET), and is available from The Boots Company Ltd., Electron Avanue, Isando 1600, South Africa.

In an alternate embodiment, the microbicidal formulation or agent of the present invention may include Zinc Undecenoate. Zinc Undecenoate is an anti-fungal agent, which is present in products such as Mycota cream, available from The Boots Company (South Africa).

Preferably, the present method involves the topical application of the microbicidal agent or formulation. In the context of the present invention, it is to be understood that the term topical application includes application to the body cavities as well as to the skin. Thus, in a preferred embodiment, the microbicidal agent or formulation is applied to a body cavity such as the vagina, anus, or mouth.

In a particularly preferred embodiment, the composition including the microbicidal agent or formulation is applied to the vagina. In a preferred embodiment, the topical application is carried out prior to the beginning of vaginal intercourse, preferably from 0 to 8 hours, more preferably from 0 to 60 minutes, and most preferably from 0 to 15 minutes. The composition including the microbicidal agent or formulation can also be used independent from intercourse.

The concentration of microbicidal agent in a formulation will vary depending on the particular agent or formulation and the base or carrier. Preferably the concentration will fall within the parameters of approximately 0.01% to approximately 50% by weight, more preferably between approximately 0.1% to approximately 28%, and most preferably between approximately 0.5% and approximately 5%. The microbicidal agent or formulation may be applied to the vagina in a number of forms including but not limited to foam, cream, wash, gel, suppository, ovule, lotion, ointment, film, tablet, foaming tablet, tampon, vaginal spray, aerosol or other base or carrier. In the case of a vaginal or rectal microbicidal formulation, the formulation normally contains a base or carrier, and is applied in its totality. In the case of a microbicidal agent, it is preferable that the composition further includes a base or carrier.

The composition containing the microbicidal agent or formulation may be applied to the vagina in any conventional manner, as would be known to one skilled in the relevant art. Suitable devices for applying the composition to the vagina are disclosed in U.S. Pat. Nos. 3,826,828, 4,108,309, 4,360,013, and 4,589,880, which are incorporated herein by reference. In a preferred embodiment, the microbicidal composition may be applied to the vagina by an applicator. In a preferred embodiment, the applicator may be a tube from approximately 2.5 to approximately 25 centimeters in length, or more preferably approximately 5 to approximately 10 centimeters in length. In an alternate embodiment, the applicator may have one or more holes distributed regularly along its length. In alternate embodiments, the applicator may be a vaginal ring, or other slow release applicators, as would be known to one skilled in the relevant art. In an alternate embodiment, the microbicidal composition may be provided in the form of a suppository, preferably a vaginal suppository.

In alternate embodiments, the microbicidal composition of the present invention may be topically administered to the rectum or anorectal area. The composition may be applied to the anus in a number of forms including but not limited to a foam, cream, jelly, or other application such as those described above with regard to vaginal application. In the case of anorectal application, it may be preferred to use an applicator, which distributes the composition substantially evenly throughout the anus. In a preferred embodiment, the applicator is a tube approximately 2.5 to approximately 25 cm in length, or more preferably approximately 5 to approximately 10 cm in length, and may include one or more holes distributed regularly along its length. In alternate embodiments, differing varieties of applicators could be used, or no applicator may be used, as would be known to one skilled in the relevant art.

In an alternate embodiment, a composition containing a microbicidal agent or formulation may be delivered orally. Oral application is preferably carried out by providing the microbicidal composition in the form of a mouthwash or gargle. In one embodiment, oral application may be used to prevent infection during dental procedures. Suitably, the microbicidal composition is applied prior to the beginning of the dental procedure and periodically throughout the procedure. In the case of a mouthwash or gargle, it may be preferred to include in the composition an agent which will mask the taste and/or odor of the microbicidal agent or formulation. Such agents include those flavoring agents typically found in mouthwashes and gargles, such as spearmint oil, cinnamon oil, or other flavoring agents.

In alternate embodiments, the microbicidal compound of the present invention may further include a vaginal contraceptive agent. Compositions including vaginal contraceptive agents are disclosed in U.S. Pat. Nos. 2,149,240, 2,330,846, 2,436,184, 2,467,884, 2,541,103, 2,623,839, 2,623,841, 3,062,715, 3,067,743, 3,108,043, 3,174,900, 3,244,589, 4,093,730, 4,187,286, 4,283,325, 4,321,277, 4,368,186, 4,371,518, 4,389,330, 4,415,585, and 4,551,148, which are incorporated herein by reference, and the present method may be carried out by applying the microbicidal agent or formulation to the vagina in the form of such a composition. Vaginal contraceptive agents are preferably not cytotoxic, such as cellulose sulfate or polystyrene sulfonate. However, cytotoxic contraceptive agents may also be used, such include nonylphenoxypolyoxyethylene glycol (nonoxynol 9), benzalkonium chloride, and chlorindanol. Suitably, the pH of the microbicidal composition is between approximately 3 to approximately 9, more preferably between approximately 3 to approximately 6, and most preferably approximately 3.5.

The present compositions may also be in the form of a time-release composition and slow-releasing devices. In such an embodiment, the microbicidal agent or formulation is incorporated in a composition that will release the agent or formulation at a rate that will result in the vaginal or anorectal concentrations described above. Time-release and slow-release compositions are disclosed in U.S. Pat. Nos. 5,185,155; 5,248,700; 4,011,312; 3,887,699; 5,143,731; 3,640,741; 4,895,724; and 4,795,642, all of which are incorporated herein by reference.

The present compositions may be provided in a form that releases the microbicidal agent or formulation in response to an event such as vaginal or anorectal intercourse. For example, the microbicidal composition may contain the microbicidal agent or formulation in vesicles or liposomes which are disrupted by the mechanical action of intercourse. Examples of compositions comprising liposomes are described in U.S. Pat. No. 5,231,112 which is incorporated herein by reference.

It should also be understood that the microbicidal compositions of the present invention may be associated with a device, such as an intrauterine device (IUD), vaginal dispenser vaginal ring, intravaginal barrier-type device, intravaginal sponge, or a male or female condom. In the case of an IUD or diaphragm, time-release and/or mechanical-release compositions may be preferred, while in the case of condoms, mechanical-release compositions may be preferred. In alternate embodiments, the device may be an intravaginal sponge that comprises and releases the microbicidal agent or formulation. Intravaginal sponges are disclosed in U.S. Pat. Nos. 3,916,898 and 4,360,013, which are incorporated herein by reference.

The device may also be a vaginal dispenser that releases a microbicidal formulation. Vaginal dispensers are disclosed in U.S. Pat. No. 4,961,931, which is incorporated herein by reference. The device may be an intravaginal barrier-type device, such as those described in U.S. Pat. Nos. 4,858,624, 4,989,618, and 5,207,232, which are incorporated herein by reference.

The device may also be a condom that is coated with a microbicidal agent or a microbicidal formulation may be incorporated in the condom. In a preferred embodiment, the microbicidal agent or formulation is encapsulated in liposomes such that the microbicidal agent or formulation is released from the liposomes upon intercourse. The condom may be coated with other lubricants and penetration-enhancing agents such as those described in U.S. Pat. Nos. 4,537,776; 4,552,872; 4,557,934; 4,130,667, 3,989,816; 4,017,641; 4,954,487; 5,208,031; and 4,499,154, which are incorporated herein by reference. In an alternate embodiment, the microbicidal agent or formulation may be contained inside the condom.

The size of the microbicidal composition will vary depending on the type of composition used. For example, when the composition is in the form of a suppository (including vaginal and anorectal suppositories), the suppository will usually be approximately 1 to approximately 5 grams, preferably approximately 3 grams, and the entire suppository will be applied. A vaginal or anorectal tablet will suitably be approximately 1 to approximately 5 grams, preferably approximately 2 grams, and the entire tablet will be applied. When the composition is vaginal cream, approximately 0.1 grams to approximately 10 grams may be applied, more preferably approximately 0.5 grams to approximately 5 grams, and most preferably approximately 3 grams to approximately five grams. When the composition is vaginal or anorectal gel, approximately 0.1 grams to approximately 10 grams may be applied, more preferably approximately 0.5 grams to approximately 5 grams, and most preferably approximately 3 grams to approximately 5 grams. When the composition is a vaginal foam, approximately 0.1 to approximately 5 grams of the spray-foam may be applied, preferably approximately 0.5 grams to approximately three grams. When the composition is an anorectal cream, approximately 0.1 to approximately 5 grams may be applied, preferably approximately 0.5 grams to approximately 3 grams, most preferably approximately 2 grams to approximately 3 grams. When the composition is an anorectal foam, approximately 0.1 ml to approximately 10 ml of the spray-foam may be applied, more preferably approximately 3 ml to approximately 8 ml, and most preferably approximately 6 ml to approximately 7 ml. When the composition is a mouthwash or gargle, approximately 1 ml to approximately 20 ml may be applied, preferably approximately 8 ml to 10 ml, and most preferably approximately 10 ml.

### EXAMPLES

### Example 1—HIV Inhibition

Ciclopirox olamine (CO) is an effective inhibitor of several different HIV strains with different host cells. Table 1 shows some sample results obtained in HIV inhibition assays conducted as described below. In a first series of assays, the compound showed a 50% effective concentration (IC₅₀) of 1.46 μg/ml when mixed with virus (HIV-I_{RF}) and host cells, followed by incubation. The IC₅₀ was about the same (0.96 μg/ml) when the compound was mixed with host cells, followed by washing and then addition of virus. A second series of assays showed IC₅₀ values of 45.5 and 6.9 μg/ml with HIV-IIIB and BaL, respectively, in the viral binding inhibition assay, and an IC₅₀ of 97.4 μg/ml in the cell-to-cell transmission assay.

Assays listed in Table 1 are described below:

PC assay: Indicator T cells (PM-1; 10⁵/well) were pre-treated with compound for 1 hour at 37° C., compound was removed by washing with 4 volumes of PBS, treated cells were added to immobilized virus (HIV-I_{RF}; 10³ TCID₅₀) and incubated for 8 days.

PV-L assay: Immobilized virus (HIV-I_{RF}; 10³ TCID₅₀) was mixed with compound for 1 hour at 37° C. before addition of indicator T cells (PM-1; 10⁵/well) and incubated for 8 days (no washing).

In each of the assays above, viral replication was monitored after 8 days by measuring reverse transcriptase activity.

Viral entry inhibition assay (VBI): Cell-free HIV-1 (strain III-B: X4 strain; or strain BaL: R5 strain) was incubated with compound and P4-R5 cells for 2 hours at 37° C., followed by washing and 48 hour incubation.

Cell to cell transmission assay (CTC): HIV-1 infected SupTi cells (strain IIIB), killed prior to exposure, were incubated with compound and P4-R5 cells for 2 hours at 37° C., followed by washing and 48 hour incubation.

Cell-free inactivation assay: Virus was incubated with product for 2 minutes, a series of 10-fold dilutions were made, each dilution was added to the host cells and viral infectivity was measured.

TABLE 1
HIV Inhibition

| | | 50% Effective |
|---|---|---|
| | HIV Strains | Concentrations |
| Series 1 | PC: HIV-I_{RF} [1 hour pre-incububation] | 0.96 μg/ml |
| | PV-L: HIV-I_{RF} [1 hour pre-incubation] | 1.46 μg/ml |
| Series 2 | VBI: IIIB [2 hour incubation] | 45.5 μg/ml |
| | VBI: BaL [2 hour incubation] | 6.9 μg/ml |
| | CTC [2 hour incubation] | 97.4 μg/ml |
| | Cell-free inactivation assay | At 0.3%: ≧2.7 log |
| | | reduction |
| | | At 0.09%: ≧2.0 log |
| | | reduction |

### Example 2—Inhibition of Other Pathogens

CO is also an effective inhibitor of various other pathogens. Table 2 shows sample results obtained from inhibition assays performed using various pathogens.

Herpes-2 Inhibition Assays

A. Viral Binding Inhibition Assay

Confluent Vero cells were incubated with different concentrations of ciclopiroxolamine at 37° C. for 1 hour. 200 pfu of HSV-2 were then added to the cells in the presence of test compound and the cells were incubated at 37° C. for 1 hour. The infected cells were washed with PBS and overlaid with methylcellulose (no drug) for a plaque assay.

B. Virucidal Assay

The virucidal activities of ciclopiroxolamine against HSV-2 were tested by mixing various concentrations of the test compound with 2×10⁵ PFU of HSV-2, respectively, and incubating at 37° C. for 60 min. The culture medium was included as a positive control. At the end of incubation, the mixtures were diluted 1 in 1000 with culture medium. The infectivity of treated virus was determined by a standard viral plaque assay. Briefly, confluent Vero cells were washed with PBS and subsequently infected with 1 ml of treated mixture at 37° C. for 1 h. After viral inoculum was removed, the infected cells were washed with PBS and overlaid with 0.5% methylcellulose in culture medium. The infected cells were incubated at 37° C. for 2 days to allow the plaques to develop.

Neisseria gonorrhea inhibition assay

Compound was incorporated into gonococcal agar, the agar/agent mixture was poured on culture dishes and inoculated with N. gonorrheae, strain ms11a (10 μl of each dilution: 10⁻¹ to 10⁻⁵ of a starting 0.5 Mcfarland solution which is equal to 1.5×10⁸ colony forming units (cfu)/ml). The plates were incubated overnight (20-22 hours) and the colonies counted.

Chlamydia trachomatis inhibition assay

C. trachomatis Serovar E elementary bodies (EGs; 10⁴ IFU) were incubated for 4 hours on ice with compound, the mixture was inoculated into HeLa cell monolayers, and incubated for 1 hour. The inoculum was removed, the medium changed and the cells incubated overnight (20-22 hours). The cells were removed, fixed, permeabilized, and the infected cells detected by fluorescently tagged antibody and flow cytometry.

Anaerobe and aerobe inhibition assays (bacteria; fungi)

Working in an anaerobic chamber, the anaerobes were suspended into brucella broth to the turbidity of the 0.5 McFarland standard and pipetted into the wells of the Steers replicator head, removed from the chamber and stamped onto the supplemented Brucella agar plates containing the serial dilutions of the antimicrobials and the growth control plates. The final inoculum was 10⁵ cfu/spot.

The aerobic organisms were suspended into saline to the turbidity of the 0.5 McFarland standard, diluted 1:10, pipetted into the wells of the Steers replicator head, and stamped onto the Mueller Hinton agar plates containing the serial dilutions of the antimicrobials.

The anaerobes were incubated in an anaerobic chamber at 37° C. for 2 days; aerobes were incubated in ambient air at 36° C. for 20h.

CO is a highly effective inhibitor of gonococci with an 50% effective concentration (IC₅₀) of 1 μg/ml. The compound also has some effect on chlamydia, with an IC₅₀ of 1.6 mg/ml. CO has an IC₅₀ of 153 μg/ml in the HSV viral binding inhibition assay and shows some virucidal activity towards this virus (40% inhibition at 400 μg/ml, the highest concentration tested). CO is a very effective inhibitor of all anaerobes tested, showing minimal inhibitory concentrations (MICs) ranging from 40-80 μg/ml towards Gardnerella vaginalis, Mobiluncus spp., Bacteroides spp., Clostridium perfringes, Fusobacterium nucleatum, Peptostreptococcus spp., Porphyromonas spp., and Prevotella spp. The compound also inhibits aerobes effectively, including E. coli, K1 pneumoniae, Pseudomonas spp., Listeria monocytogenes, Enterococcus spp., Streptococcus spp. and Staphylococcus aureus, with MICs ranging from 40 to 160 μg/ml. Finally, it is an excellent inhibitor of Candida albicans and Candida glabrata with an MIC of 20 μg/ml.

TABLE 2
Inhibition of Other Pathogens

| Pathogens | Assays | Effective Concentrations |
|---|---|---|
| HSV-2 | Viral binding | IC₅₀ of 153 μg/ml in the viral |
| | inhibition assay | binding inhibition assay |
| | Virucidal assay | IC₅₀ of About 40% inhibition at |
| | | 400 μg/ml in the virucidal assay |
| Gonococci | Neisseria | IC₅₀ of 1 μg/ml |
| | gonorrhea | |
| | inhibition assay | |
| Chlamydia | Chlamydia | IC₅₀ of 1,631 μg/ml |
| | trachomatis | |
| | inhibition assay | |
| Candida spp. | Aerobe | Minimum Inhibitory |
| | inhibition assay | Concentrations (MICs): |
| | | C. albicans (2 strains): |
| | | 20 μg/ml each |
| | | C. glabrata (3 strains): |
| | | 20 μg/ml each |
| Bacteria | Anaerobe | Minimum Inhibitory |
| | and aerobe | Concentrations (MICs): |
| | inhibition assay | Anaerobes: |
| | | Gardnerella vaginalis (2 strains): |
| | | 80 μg/ml each |
| | | Mobiluncus curtisii (2 strains): |
| | | 40 μg/ml each |
| | | Mobiluncus mulieris (2 strains): |
| | | 40 μg/ml each |
| | | Bacteroides fragilis (1 strain): |
| | | 40 μg/ml |
| | | Bacteroides thetaiotaomicron (1 |
| | | strain): 40 μg/ml |
| | | Clost perfringes (2 strains): |
| | | 40 μg/ml each |
| | | Eubact lentum (2 strains): |
| | | 40 μg/ml each |
| | | Fuso. nucleatum (2 strains): |
| | | 40 μg/ml |
| | | Peptostrep anaerobius (1 strain): |
| | | 40 μg/ml |
| | | Peptostrep asaccharolyticus (2 |
| | | strains): 40 and 80 μg/ml |
| | | Peptrostrep. magnus (2 strains): |
| | | 40 μg/ml each |
| | | Peptostrep tetradius (1 strain): |
| | | 40 μg/ml |
| | | Porhyromonas asaccharolytica (2 |
| | | strains): 40 μg/ml each |
| | | Porphyromonas levii (2 strains): |
| | | 40 μg/ml each |
| | | Prevotella bivia (2 strains): |
| | | 40 μg/ml each |
| | | Prevotella disiens (2 strains): |
| | | 40 μg/ml each |
| | | Prevotella intermedia (2 strains): |
| | | 40 μg/ml each |
| | | Prevotella melaninogenica (2 |
| | | strains): 40 μg/ml each |
| | | Aerobes |
| | | E. coli (2 strains): |
| | | 40 and 80 μg/ml |
| | | K1 pneumoniae (2 strains): |
| | | 40 and 80 μg/ml |
| | | Pseudo aerug (1 strain): |
| | | 80 μg/ml |
| | | Pseudo fluorescens (1 strain): |
| | | 160 μg/ml |
| | | Listeria monocytogenes (2 |
| | | strains): |
| | | 40 μg/ml each |
| | | Enterococcus faecalis (1 strain): |
| | | 80 μg/ml |
| | | Enterococcus faecium (1 strain): |
| | | 40 μg/ml |
| | | Strep pyogenes (2 strains): |
| | | 80 μg/ml each |
| | | Strep agalactiae (2 strains): |
| | | 80 and 160 μg/ml |
| | | Staph. aureus (3 strains): |
| | | 40, 40 and 80 μg/ml |

### Example 3—Safety and Toxicity

Tables 3, 4, and 5 show some sample results of safety and toxicity assays performed using CO. In some samples tested, CO was complexed with cyclodextrin (CD). CO inhibits the growth of lactobacilli but does not kill them, presumably allowing rapid re-colonization if growth of this microbe is impeded while a CO formulation is in the vagina. It is not overtly cytotoxic to host cells in vitro; the toxicity varies with the cell type. Towards HeLa cells, a first series of assays show a cytotoxic concentration 50% (CC₅₀) of >100 μg/ml and another of 859 μg/ml. The CC₅₀ towards Vero cells is >200 μg/ml, towards P4-R5 cells >100 μg/ml and towards VK-2/E6E7 cells 100 μg/ml. A 1% CO cream is safe in the rabbit vaginal irritation assay when applied for 10 consecutive days with an overall score of 3.93.

Assays listed in Table 3 are described below:

Lactobacillus assays:

A. Macrobroth Dilution (MBD) Assay

Ciclopiroxolamine was serially diluted in brucella broth supplemented with laked sheep blood, vitamin K, and hemin (the highest concentration tested was 10 mg/mL). Aliquots (lmL) of the mixtures were added to 12×75-mm plastic tubes. Test lactobacillus strains were suspended to a turbidity equal to the one-half McFarland standard in supplemented brucella broth and diluted ˜1:50 in brucella broth to obtain a concentration of ˜3×10⁶ colony forming units (CFU)/mL. Samples (1 mL) of the test strains were added to the ciclopiroxolamine dilution series and then incubated for 2 days at 37° C. under anaerobic conditions. Tubes containing no organism or no drug were tested simultaneously as negative and positive controls. Minimum inhibitory concentration (MICs) were recorded.

B. Agar Dilution (AD) Assay

Serial dilutions of ciclopiroxolamine were mixed with molten brucella agar supplemented with lysed sheep blood, vitamin K, and hemin (NCCLS reference agar dilution method) and poured into separate plates. After the plates were solidified and dried, suspensions of the test organism (10⁸ CFU/mL) were spotted onto the surface, using a replicating device that delivered a final concentration of 10⁵ CFU/spot. After incubation at 37° C. for 48 hours under anaerobic conditions, the plates were examined for growth. MICs were recorded.

C. Steers Time-Kill (TK) Assay

The TK assay was performed with ciclopiroxolamine that did not produce a clear solution in the broth medium. A working stock of the formulation was mixed 1:10 with brucella broth and pipetted into wells of the replicator. Organisms were added at a concentration of 5×10⁶ to 1×10⁷ CFU/mL, incubated in an anaerobic chamber at 37° C., and subcultured hourly to blood agar plates. The subculture plates were incubated for 2-3 days and examined for growth. The results are expressed in hours of survival.

Cytotoxicity Assays

The cells listed in Table 4 were incubated for the indicated period of time with different concentrations of ciclopiroxolamine and cell viability was measured by the 3-(4,5-dimethylthiazol-2-yl)-2,5-diphenyltetrazolium bromide (MTT) assay or by treatment with propidium iodide and fluorescence. Percent viability was calculated with respect to the viability of mock-exposed cells (cells incubated without compounds). Table 4 shows the cytotoxic concentration 50% (CC₅₀) values obtained.

In the MTT assay, the effect of each agent on cell viability was determined using an assay in which dehydrogenases in viable cells cleave the compound 3-(4,5-dimethylthiazol-2-yl)-2,5-diphenyltetrazolium bromide (MTT), yielding a purple product (formazan) which can be measured spectrophotometrically (Pauwels et al., 1988). Cells were seeded into 96 well plates at a density of 3×10⁴ cells/well 24 hours prior to introduction of the microbicides. Agents were diluted in as previously indicated. Agents were added to triplicate wells in half-log serial dilutions. Cells were incubated in the absence or presence of each agent at 37° C. under 5% CO₂ and 90% humidity for 2 hours. At the conclusion of each experiment, cells were washed, replenished with fresh medium and incubated with MTT (250 μl/well) for 2 hours at 37° C. Following removal of the medium (by aspiration), intracellular formazan crystals were solubilized with 10% Triton X-100 in acidified isopropanol. The resulting solutions were assayed spectrophotometrically at 570 nm and corrected for non-specific absorption at 690 nm. In this assay, measured absorbance is proportional to the viable cell number, and inversely proportional to the degree of cytotoxicity.

Rabbit Vaginal Irritation Assay

Mature female New Zealand White rabbits were divided into three test groups. One group served as untreated control. The other two groups were treated vaginally for 10 consecutive days with either the CO formulation (1 mL per rabbit per day) or served as sham control (insertion of applicator without gel). All animals were weighed on the first day of dosing and every seventh day thereafter. The animals were observed at least twice daily for moribundity/mortality. Observations were also made for vaginal bleeding and discharges, appearance, behavior, and pharmacotoxic signs, approximately one half hour and four hours after dosing. Detailed physical observations were performed daily. The animals were necropsied 24 hours after the final vaginal dose of gel was administered and the vagina collected, fixed, sectioned and studied histopathologically. Results are shown in Table 5.

Safety

TABLE 3
Lactobacilli

| | | MIC Values |
|---|---|---|
| | Original | |
| | material: | |
| L. jensii | Macrobroth | MIC of between 20 and 100 μg/ml |
| (2 strains); | dilution assay | except for one strain: >100 μg/ml |
| | Complexed | |
| | with CD: | |
| L. gasseri | Agar | pH 7.2: MIC from 125 to 1,000 μg/ml |
| (3 strains); | dilution | |
| | assay | |
| L. acidophilus | | pH 4.8: MIC from 60 to 125 μg/ml |
| (2 strains); | | |
| L. salivarius | Steers time | No kill at 1,000 μg/ml (highest |
| (2 strains). | kill assay | dose tested) in 24 hours (longest |
| | | period tested) except for two |
| | | strains: |
| | | >8 <23 hours. |
| | Steers time | At 10,000 μg/ml: |
| | kill assay | L. crispatus (2 strains): >24 hrs |
| | | L. salivarius (2 strains): >8<24 hrs |
| | | L. gasseri (3 strains): 3; 3; >24 hrs |
| | | L. jensii (2 strains): <1; 2 hrs |

TABLE 4
Cytotoxicity

| | | |
|---|---|---|
| Series 1 | Original material: | CC₅₀ of |
| | HeLa [1 hour incub] | >100 μg/ml (highest conc tested) |
| Series 2 | P4-R5 [CTS; | CC₅₀ of |
| | 2 hour incubation] | >100 μg/ml (highest conc tested) |
| | VK-2/E6E7 [vaginal | CC₅₀ of 100 μg/ml |
| | epithelium cell line; | |
| | 2 hour incubation] | |
| Series 3 | HeLa [1 hour incubation] | CC₅₀ of 859 μg/ml |
| Series 4 | Vero [2 hour incubation] | CC₅₀ of >200 μg/ml |

TABLE 5
Rabbit vaginal irritation assay

| | |
|---|---|
| Olamin cream (contains 1% ciclopirox). | Untreated control: 1.1; |
| 10 consecutive days of vaginal application | Sham: 1.3; |
| (Scores) | Olamin: 3.93 |

### Example 4—Sperm Inhibition and Condom Stability

CO has very little cytotoxicity towards spermatozoa and would not be considered spermicidal. Short term incubation with the condom (1 hour) but longer than the expected time of contact during coitus, has no effect on burst pressure or burst volume. Tables 6 and 7 show some sample results obtained form sperm inhibition and condom stability assays.

The assays listed in Table 6 and 7 are described below:

Sperm Immobilization Assay

To assess sperm-immobilizing activity, compounds were incubated with sperm for 30 seconds in two fold serial dilutions. Those dilutions that immobilized all screened spermatozoa were further diluted several times in buffer and incubated at 37° C., 5% CO₂, for 1 h, to verify any possible recovery of motility. Only those dilutions (compound concentrations) that immobilized all spermatozoa with no recovery of motility were considered “positive” and used to calculate the compound's minimum effective concentration (MEC).

Condom Stability Assay

The Airburst Test Method was used to test condom stability. Both volume and pressure were recorded. Baseline testing was performed using 50 randomly selected condoms to establish a baseline. Control testing was performed using 50 randomly selected condoms. Prior to the control testing, the condoms were treated with a water based lubricant such as KY Jelly. Sample testing was performed using 50 randomly selected condoms. Prior to the sample testing, the condoms were treated with ciclopiroxolamine. A pre-determined quanitity of the ciclopiroxolamine or a water based lubricant were applied to each condom using either a brush or sponge, insuring that the ¾ of the condom, measured from the closed end, is completely coated. Following the application, the condoms were placed in an oven at 37° C. for one hour. Following oven conditioning, the ciclopiroxolamine or water based lubricant was carefully removed from the condoms using a soft towel (or cheese cloth). Caution was taken not to stretch or place unnecessary stress on the condom. The condoms were then airbursted immediately.

TABLE 6
Sperm Inhibition

| | |
|---|---|
| Sperm immobilization activity | MEC: 1,200-8,800 μg/ml |

TABLE 7
Condom Stability

| | |
|---|---|
| Incubation for 1 hour at 37° C. Measure | |
| burst pressure and burst volume. | |
| Solution of 1%, 0.1% or 0.01% ciclopirox | No effect as compared to |
| with cyclodextrin. | cyclodextrin control. A |
| | −4% to +4.5% change as |
| | compared to KY jelly. |
| Olamin cream (contains 1% ciclopirox) | No effect. A 3-7% decrease |
| | as compared to KY jelly. |

While the invention has been particularly shown and described with reference to preferred embodiments thereof, it will be understood by those skilled in the art that various changes in form and details may be made therein without departing from the spirit and scope of the invention.

1. A microbicidal composition for preventing the transmission of a sexually transmitted infection comprising at least one microbicidal agent, wherein said at least one microbicidal agent comprises ciclopirox olamine.

2. The microbicidal composition of claim 1, further comprising a carrier selected from the group consisting of foam, cream, wash, gel, suppository, ovule, lotion, ointment, film, tablet, foaming tablet, tampon, vaginal spray and aerosol.

3. The microbicidal composition of claim 1, wherein said ciclopirox olamine is a white crystal powder.

4. The microbicidal composition of claim 1, wherein said ciclopirox olamine has the formula C₁₂H₁₇NO₂C₂H₇NO.

5. The microbicidal composition of claim 1, comprising about 0.01% to about 50% by weight of said microbicidal agent.

6. The microbicidal composition of claim 5, comprising about 0.1% to about 28% by weight of said microbicidal agent.

7. The microbicidal composition of claim 6, comprising about 0.5% to about 5% by weight of said microbicidal agent.

8. The microbicidal composition of claim 1, further comprising a vaginal contraceptive agent.

9. The microbicidal composition of claim 1, having a pH of between about 3 to about 9.

10. The microbicidal composition of claim 9, having a pH of between about 3 to about 6.

11. The microbicidal composition of claim 10, having a pH of about 3.5.

12. The microbicidal composition of claim 1, wherein said composition is a time-release composition.

13. The microbicidal composition of claim 1, wherein said sexually transmitted infection is selected from the group consisting of HIV/AIDS, herpes, gonorrhea, chlamydia, syphilis, and trichomoniasis.

14. The microbicidal composition of claim 1, further comprising an applicator.

15. A device comprising the microbicidal composition of claim 1, wherein said device is selected from the group consisting of intrauterine device, vaginal dispenser, vaginal ring, intravaginal barrier-type device, intravaginal sponge, male condom, and female condom.

16. A method of preventing the transmission of a sexually transmitted infection comprising topically applying a microbicidal composition comprising at least one microbicidal agent, wherein said microbicidal agent comprises ciclopirox olamine.

17. The method of claim 16, comprising applying said microbicidal composition to the skin.

18. The method of claim 16, comprising applying said microbicidal composition to a body cavity.

19. The method of claim 18, wherein said body cavity is the mouth.

20. The method of claim 18, wherein said body cavity is the anus.

21. The method of claim 18, wherein said body cavity is the vagina.

22. The method of claim 21, wherein said microbicidal composition is applied prior to the beginning of vaginal intercourse.

23. The method of claim 22, wherein said microbicidal composition is applied between about 0 and about 8 hours prior to the beginning of vaginal intercourse.

24. The method of claim 23, wherein said microbicidal composition is applied between about 0 and about 60 minutes prior to the beginning of vaginal intercourse.

25. The method of claim 24, wherein said microbicidal composition is applied between about 0 to about 15 minutes prior to the beginning of vaginal intercourse.

26. The method of claim 21, wherein said microbicidal composition is applied independent of intercourse.

27. The method of claim 16, wherein said microbicidal composition is in a form selected from the group consisting of foam, cream, wash, gel, suppository, ovule, lotion, ointment, film, tablet, foaming tablet, tampon, vaginal spray and aerosol.

28. The method of claim 16, comprising applying said microbicidal composition to the rectum.

29. The method of claim 16, comprising applying said microbicidal composition to the anorectal area.

30. The method of claim 16, further comprising using a contraceptive device.

31. The method of claim 30, wherein said contraceptive device is selected from the group consisting of intrauterine device, intravaginal barrier-type device, intravaginal sponge, male condom, and female condom.

32. The method of claim 27, comprising applying about 1 to about 5 grams of said suppository.

33. The method of claim 32, comprising applying about 3 grams of said suppository.

34. The method of claim 27, comprising applying about 1 to about 5 grams of said tablet.

35. The method of claim 34, comprising applying about 2 grams of said tablet.

36. The method of claim 27, comprising applying about 0.1 to about 10 grams of said cream wherein said cream is a vaginal cream.

37. The method of claim 36, comprising applying about 0.5 to about 5 grams of said cream wherein said cream is a vaginal cream.

38. The method of claim 37, comprising applying about 3 to about 5 grams of said cream wherein said cream is a vaginal cream.

39. The method of claim 27, comprising applying about 0.1 to about 10 grams of said gel.

40. The method of claim 39, comprising applying about 0.5 to about 5 grams of said gel.

41. The method of claim 40, comprising applying about 3 to about 5 grams of said gel.

42. The method of claim 27, comprising applying about 0.1 to about 5 grams of said foam wherein said foam is a vaginal foam.

43. The method of claim 42, comprising applying about 0.5 to about 3 grams of said foam wherein said foam is a vaginal foam.

44. The method of claim 27, comprising applying about 0.1 to about 5 grams of said cream wherein said cream is an anorectal cream.

45. The method of claim 44, comprising applying about 0.5 to about 3 grams of said cream wherein said cream is an anorectal cream.

46. The method of claim 45, comprising applying about 2 to about 3 grams of said cream wherein said cream is an anorectal cream.

47. The method of claim 27, comprising applying about 0.1 to about 10 ml of said foam wherein said foam is an anorectal foam.

48. The method of claim 47, comprising applying about 3 to about 8 ml of said foam wherein said foam is an anorectal foam.

49. The method of claim 48, comprising applying about 6 to about 7 ml of said foam wherein said foam is an anorectal foam.

50. The method of claim 16, wherein said microbicidal agent is released upon sexual intercourse.

51. The method of claim 50, wherein said microbicidal agent is contained in vesicles.

52. The method of claim 50, wherein said microbicidal agent is contained in liposomes.

53. The method of claim 16, wherein said microbicidal composition comprises about 0.01% to about 50% by weight of said microbicidal agent.

54. The method of claim 16, wherein said sexually transmitted infection is selected from the group consisting of HIV/AIDS, herpes, gonorrhea, chlamydia, syphilis, and trichomoniasis.

55. A method of treating a sexually transmitted infection comprising topically applying a microbicidal composition comprising at least one microbicidal agent, wherein said microbicidal agent comprises ciclopirox olamine.

56. The method of claim 55, comprising applying said microbicidal composition to the skin.

57. The method of claim 55, comprising applying said microbicidal composition to a body cavity.

58. The method of claim 57, wherein said body cavity is the vagina.

59. The method of claim 55, wherein said microbicidal composition is in a form selected from the group consisting of foam, cream, wash, gel, suppository, ovule, lotion, ointment, film, tablet, foaming tablet, tampon, vaginal spray and aerosol.

60. The method of claim 55, wherein said microbicidal composition comprises about 0.01% to about 50% by weight of said microbicidal agent.

61. The method of claim 55, wherein said sexually transmitted infection is selected from the group consisting of HIV/AIDS, herpes, gonorrhea, chlamydia, syphilis, and trichomoniasis.

62. A method of preventing the transmission of a common vaginal infection comprising topically applying a microbicidal composition comprising at least one microbicidal agent, wherein said microbicidal agent comprises ciclopirox olamine.

63. The method of claim 62, comprising applying said microbicidal composition to the skin.

64. The method of claim 62, comprising applying said microbicidal composition to the vagina.

65. The method of claim 62, wherein said microbicidal composition is in a form selected from the group consisting of foam, cream, wash, gel, suppository, ovule, lotion, ointment, film, tablet, foaming tablet, tampon, vaginal spray and aerosol.

66. The method of claim 62, wherein said microbicidal composition comprises about 0.01% to about 50% by weight of said microbicidal agent.

67. The method of claim 62, wherein said common vaginal infection is bacterial vaginosis.

68. The method of claim 62, wherein said common vaginal infection is vaginal candidiasis.

69. A microbicidal composition for preventing the transmission of a sexually transmitted infection comprising at least one microbicidal agent, wherein said at least one microbicidal agent is a hydroxypyridone.
